(19) United States
(12) Patent Application Publication
(10) Pub. No.: US 2025/0082893 A1
(43) Pub. Date: Mar. 13, 2025

(54) PERIPHERAL SENSORY STIMULATION AND SYNCHRONIZED DEEP BREATHING IN THE TREATMENT OF DISORDERS

(71) Applicants: Leslie A. Nussbaum, St. Louis Park, MN (US); Tom Harold, Minneapolis, MN (US); Eric Nussbaum, St. Louis Park, MN (US)

(72) Inventors: Leslie A. Nussbaum, St. Louis Park, MN (US); Tom Harold, Minneapolis, MN (US); Eric Nussbaum, St. Louis Park, MN (US)

(21) Appl. No.: 18/628,052

(22) Filed: Apr. 5, 2024

Related U.S. Application Data
(60) Provisional application No. 63/581,566, filed on Sep. 8, 2023.
(60) Provisional application No. 63/584,418, filed on Sep. 21, 2023.
(60) Provisional application No. 63/590,349, filed on Oct. 13, 2023.
(60) Provisional application No. 63/624,640, filed on Jan. 24, 2024.

(51) Int. Cl. A61M 21/02; A61H 9/00; A61M 21/00

(52) U.S. Cl. CPC A61M 21/02; A61H 9/0007; A61H 9/0071; A61H 2201/025; A61H 2201/1635; A61H 2201/164; A61H 2201/1645; A61H 2201/1685; A61H 2201/5007; A61H 2205/067; A61H 2205/12; A61M 2021/0022; A61M 2021/0088

(57) ABSTRACT

The present disclosure relates to peripheral sensory stimulation and synchronized deep breathing systems, devices, and methods used in the treatment of various neurological disorders. A peripheral sensory stimulation system of the present disclosure may include a control module, a power source configured to provide electrical power to the control module, and a stimulation delivery unit coupled to the control module via tubing. The stimulation delivery unit can be configured to receive a human appendage to provide stimulation therapy for the treatment of a neurological disorder. The stimulation delivery unit can provide stimulation therapy to a user by directing air out of a plurality of apertures and onto the human appendage, the pattern of delivered air being controlled by the control module. A decrease in symptoms associated with various neurological disorders including anxiety, depression, traumatic brain injury, PTSD, and insomnia is reported, along with increasing user relaxation and satisfaction during treatment.

### CROSS-REFERENCE TO RELATED APPLICATIONS

The present application claims the benefits of U.S. Provisional Application No. 63/624,640, filed Jan. 24, 2024, U.S. Provisional Application No. 63/590,349, filed Oct. 13, 2023, U.S. Provisional Application No. 63/584,418, filed Sep. 21, 2023, and U.S. Provisional Application No. 63/581,566, filed Sep. 8, 2023, the disclosures of which are incorporated by reference herein in their entireties.

### FIELD

The present disclosure generally relates to sensory stimulation. More specifically, the present disclosure relates to peripheral sensory stimulation and synchronized deep breathing systems, devices, and methods used in the treatment of various neurological disorders.

### BACKGROUND

Anxiety, depression, and insomnia are extremely common disorders which result in a tremendous societal burden in terms of impaired quality of life, disability, medical costs, and unemployment. It has been suggested that these conditions may be associated with impaired sensory processing, suggesting that sensory stimulation could be used as a potential therapeutic option. Sensory stimulation including transcranial magnetic stimulation, auditory stimulation with music, olfactory stimulation with particular odors, and gustatory stimulation with particular foods have all been shown to improve mood and anxiety. Another common disorder is post-traumatic stress disorder (PTSD), which may develop in select individuals after exposure to extremely traumatic events. It is estimated that more than seven million individuals in the United States are diagnosed with PTSD every year, with many more cases being unreported. PTSD appears to represent a form of anxiety disorder that has been associated with hyperarousal and increased sympathetic output. In addition to pharmacotherapy and psychological interventions, treatment for PTSD has included various methods of relaxation including deep breathing exercises.

Peripheral sensory stimulation is a non-invasive technique to provide somatosensory stimulation to the brain which may be beneficial to users with a variety of neurological disorders such as anxiety, depression, and PTSD. Animal models have shown that early peripheral sensory stimulation significantly and reproducibly improves neurological outcomes following ischemic injury and may prevent injury entirely if applied early. Potential mechanisms for this protection and benefit include encouragement of collateral blood supply to the affected sensorimotor cortex to improve regional cerebral perfusion and/or neuronal reorganization allowing for heightened functional recovery. Clinical experience in humans has suggested that such peripheral sensory stimulation can improve recovery after medical conditions such as stroke and Parkinson's disease, and may also be beneficial following traumatic brain injury and in autoimmune conditions such as multiple sclerosis.

Improvements in peripheral sensory stimulation and synchronized deep breathing will facilitate the rehabilitation, improvement, or restoration of motion in users with neurological disorders, such as users who have experienced stroke, Parkinson's Disease, traumatic brain injuries, PTSD, and head injuries. Users who experience anxiety, depression, and insomnia can also benefit from such improvements. The present disclosure provides several solutions that improve peripheral sensory stimulation and synchronized deep breathing compared to conventional solutions.

### SUMMARY

Embodiments described or otherwise contemplated herein provide the advantage of eliminating or reducing neurological disorder symptoms using an ergonomic and safe therapy delivery unit. Typical approaches to dealing with neurological disorders may use pharmaceuticals as part of the treatment, in addition to psychological interventions by a medical practitioner. The embodiments of the present disclosure do not rely on pharmaceuticals to provide stimulation therapy, nor do they require a user to be in a medical setting to receive treatment (the user can receive treatment at home using embodiments of the present disclosure). The simple but effective embodiments provided herein use peripheral somatosensory stimulation (PSS) in the form of bursts of air and/or synchronized deep breathing to achieve neurological symptom improvement. Early clinical studies show positive improvements for users of the embodiments described herein, especially for users suffering from anxiety, depression, PTSD, insomnia, and other neurological conditions.

Among other things, the present disclosure describes systems, devices, and methods that provide PSS in the form of pneumatic puffs of air. There is evidence that early peripheral sensory stimulation may improve neurological outcomes following an ischemic stroke, in the setting of Parkinson's Disease, PTSD, and in other neurological disorders/conditions such as anxiety, depression, traumatic brain injury, and insomnia (collectively, “neurological disorder” and/or “neurological condition.” Embodiments of the present disclosure provide a measurable improvement of symptoms and quality-of-life measures in users with neurological disorders by using, for example, pneumatic puffs of air to reduce neurological symptom severity.

Certain embodiments of the present disclosure provide a chamber with multiple apertures along a chamber length to allow for puffs of air to be directed to the volar surface of a distal forearm, palm, fingers, and/or foot of a user. The disclosed embodiments are intended to provide peripheral sensory stimulation to a body part, such as a hand, through pneumatic puffs of air to encourage collateral blood supply and sensorimotor development. Embodiments may use a pneumatic pump to delivery intermittent puffs of air cycling between, for example, one second on and two seconds off from delivered air. For example, a user's hand with the palm facing up can be placed within a chamber on a gel pad to provide a comfortable resting surface for the duration of the treatment, which lasts approximately thirty minutes in certain examples (other examples have found suitable treatment in approximately fifteen minutes). Generally speaking, embodiments of the present disclosure facilitate the rehabilitation, improvement, or restoration of motion in users with neurological disorders.

In a particular embodiment, a peripheral sensory stimulation system may include a control module, a power source configured to provide electrical power to the control module, and a stimulation delivery unit coupled to the control module via tubing. The stimulation delivery unit can be configured to receive a human appendage to provide stimulation therapy for the treatment of a neurological disorder. The stimulation delivery unit can provide stimulation therapy to a user by directing air out of a plurality of apertures and onto the human appendage, the pattern of delivered air being controlled by the control module. In a related embodiment, the control module may include an integrated air compressor configured to deliver compressed air to the stimulation delivery unit. In a related embodiment, the human appendage can be at least one of a hand, one or more fingers, a forearm, and a foot. In a related embodiment, the stimulation delivery unit may include an upper housing separated from a lower housing by a gasket, the upper housing defining the plurality of apertures through which air is provided.

In a related embodiment, a top surface of the stimulation delivery unit can have a convex shape to match the convex shape of a human hand. In a related embodiment, the stimulation delivery unit can include a chamber configured to receive the human appendage, the chamber having a floor pad that the human appendage can rest on during stimulation therapy. In a related embodiment, the control module can be configured to deliver a pattern of pulsed bursts of air to the stimulation delivery unit and can be configured to control the degree of stimulation via the pattern of pulsed air bursts. In a related embodiment, the control module can be configured to deliver a pattern of continuous air to the stimulation delivery unit and can be configured to control the degree of stimulation via the pattern of continuous air. In a related embodiment, the plurality of apertures can be defined on a top surface of the stimulation delivery unit in a pattern that mimics the outline of a hand.

In a particular embodiment, a peripheral sensory stimulation system may include a control module, a power source configured to provide electrical power to the control module, a first stimulation delivery unit configured to receive a human appendage within a chamber to provide stimulation therapy for the treatment of a neurological disorder, the first stimulation delivery unit providing stimulation therapy to a user by directing air through a plurality of apertures defined in the chamber and toward the human appendage positioned inside the chamber, and a second stimulation delivery unit configured to receive a human appendage to provide stimulation therapy for the treatment of a neurological disorder, the second stimulation delivery unit providing stimulation therapy to a user by directing air out of a plurality of apertures and onto the human appendage. The first stimulation delivery unit and the second stimulation delivery unit are configured for interchangeable use with the control module.

In a related embodiment, the control module may include an integrated air compressor configured to deliver compressed air to the first stimulation delivery unit and the second stimulation delivery unit. In a related embodiment, the human appendage can be at least one of a hand, one or more fingers, and a forearm. In a related embodiment, the second stimulation delivery unit may include an upper housing separated from a lower housing by a gasket, the upper housing defining the plurality of apertures through which air is provided. In a related embodiment, a top surface of the second stimulation delivery unit can have a convex shape. In a related embodiment, the control module can be configured to deliver a pattern of pulsed bursts of air to the first and second stimulation delivery units. In a related embodiment, the control module can be configured to deliver a pattern of continuous air to the first and second stimulation delivery units. In a related embodiment, the plurality of apertures can be defined on a top surface of the second stimulation delivery unit in a pattern that mimics the outline of a hand. In a related embodiment, the control module can be configured to control the temperature of the pattern of delivered air.

In a particular embodiment, a stimulation delivery unit may include a housing configured to receive a human appendage of a user, the housing defining a plurality of apertures in contact with a plurality of air tubes, the plurality of apertures being in contact with at least a portion of the human appendage during operation. The stimulation delivery unit may further include a tunnel structure coupled to the housing, the tunnel structure configured to receive at least a portion of the human appendage, and a tubing coupling configured to receive tubing connected to an external control module, the tubing coupling further configured to transfer air from the tubing into the housing. Air can be directed from the tubing coupling into the plurality of air tubes and out of the plurality of apertures to provide stimulation therapy to the human appendage, thereby treating a neurological disorder of a user through peripheral somatosensory stimulation.

In a related embodiment, the housing can be a hollow chamber that receives the human appendage. In a related embodiment, the housing may include an upper portion having a convex surface, the plurality of apertures being defined by the upper portion, a lower portion coupled to the upper portion, the lower portion defining a plurality of channels through which air is transported, and a gasket positioned between the upper portion and the lower portion, the gasket defining a plurality of apertures corresponding to the plurality of apertures defined by the upper portion.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A and 1B are perspective views of a peripheral sensory stimulation system, according to embodiments of the present disclosure.

FIGS. 2A and 2B are top views of a stimulation delivery unit of the peripheral sensory stimulation system depicted in FIGS. 1A and 1B, showing a forearm inserted therein, according to embodiments of the present disclosure.

FIG. 3 is a perspective view of a peripheral sensory stimulation system, according to an embodiment of the present disclosure.

FIGS. 4A and 4B are top perspective and side views, respectively, of a stimulation delivery unit, according to embodiments of the present disclosure.

FIGS. 4C and 4D are bottom perspective and top views, respectively, of a stimulation delivery unit, according to embodiments of the present disclosure.

FIGS. 4E and 4F are bottom and exploded views, respectively, of a stimulation delivery unit, according to embodiments of the present disclosure.

FIG. 4G is a cross-section view of a stimulation delivery unit depicting the intersection between the upper housing, the gasket, and the lower housing, according to an embodiment of the present disclosure.

FIG. 5A is a schematic view depicting an example hole pattern of a stimulation delivery unit overlayed against a hand, according to an embodiment of the present disclosure.

FIGS. 5B and 5C are depictions of a left hand and a right hand, respectively, placed against a stimulation delivery unit, according to embodiments of the present disclosure.

FIG. 6A is a perspective view of a small stimulation delivery unit and a perspective view of a large stimulation delivery unit, according to embodiments of the present disclosure.

FIG. 6B is a side view of a large stimulation delivery unit, according to an embodiment of the present disclosure.

FIGS. 7A and 7B are top and bottom perspective views, respectively, of a stimulation delivery unit, according to embodiments of the present disclosure.

FIG. 7C is a side view of a stimulation delivery unit, according to an embodiment of the present disclosure.

FIG. 8 is a block diagram of a stimulation delivery unit, according to an embodiment of the present disclosure.

FIGS. 9A and 9B are top views of a first stimulation delivery unit designed to support five fingers, according to embodiments of the present disclosure.

FIGS. 10A and 10B are top views of a second stimulation delivery unit designed to support five fingers, according to embodiments of the present disclosure.

FIGS. 11A and 11B are top views of a third stimulation delivery unit designed to support four fingers, according to embodiments of the present disclosure.

FIGS. 12A and 12B are top views of a fourth stimulation delivery unit designed to support four fingers, according to embodiments of the present disclosure.

FIGS. 13A and 13B are top views of a fifth stimulation delivery unit designed to support four fingers, according to embodiments of the present disclosure.

FIGS. 14A-14D are top views of a sixth stimulation delivery unit designed to support four fingers, according to embodiments of the present disclosure.

FIGS. 15A-15D are top views of a seventh stimulation delivery unit designed to support four fingers, according to embodiments of the present disclosure.

FIGS. 16A and 16B are perspective and side views, respectively, of a stimulation delivery unit and a connector tube, according to embodiments of the present disclosure.

FIG. 16C is a cross-section view of the stimulation delivery unit of FIGS. 15A and 15B, according to an embodiment of the present disclosure.

FIGS. 17A and 17B are perspective and side views, respectively, of a stimulation delivery unit having a plurality of tubing couplings, according to embodiments of the present disclosure.

FIG. 18 is a perspective view of an eighth stimulation delivery unit, according to an embodiment of the present disclosure.

FIGS. 19A-19C are perspective views of a ninth stimulation delivery unit, according to embodiments of the present disclosure.

FIGS. 20A and 20B are perspective views of a tenth stimulation delivery unit, according to embodiments of the present disclosure.

FIG. 21 is a perspective view of an eleventh stimulation delivery unit, according to an embodiment of the present disclosure.

While various embodiments are amenable to various modifications and alternative forms, specifics thereof have been shown by way of example in the drawings and will be described in detail. It should be understood, however, that the intention is not to limit the claims to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the disclosure.

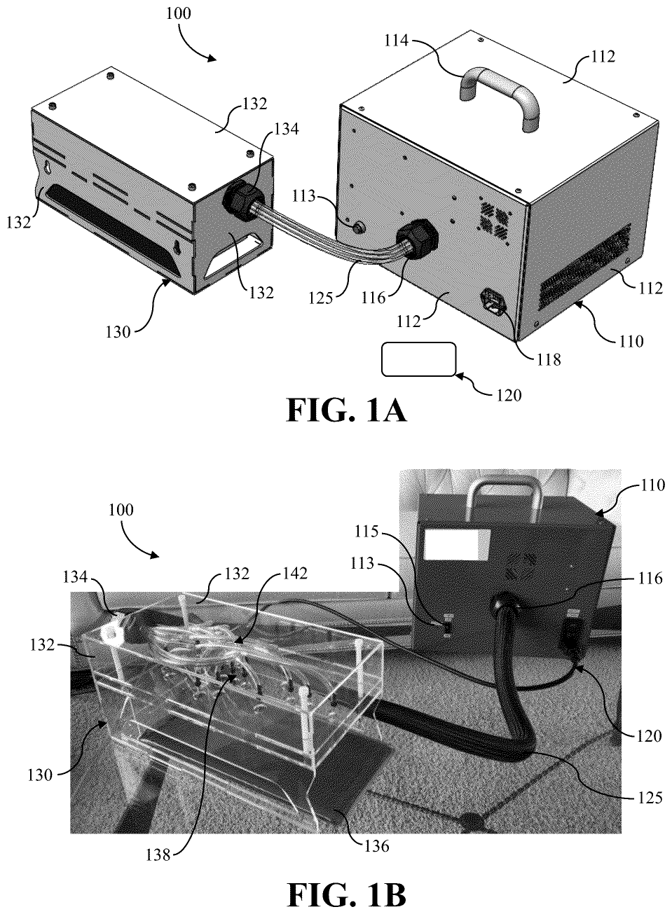

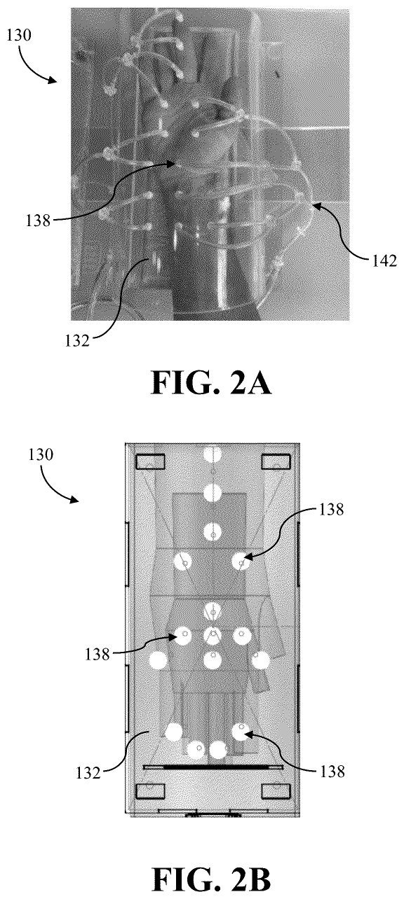

Referring generally to FIGS. 1A-2B, a peripheral sensory stimulation system 100 according to an embodiment of the present disclosure may include a control module 110 having an integrated air compressor and a housing with a plurality of sides 112, a handle 114 on a top side 112 of the control module 110, a tubing coupling 116 connectable to a side 112 of the control module 110, and a power source coupling 118 defined within a side 112 of the control module 110. The peripheral sensory stimulation system 100 may further include a power source 120, tubing 125, and a stimulation delivery unit 130 couplable to the control module 110 via the tubing 125. Stimulation delivery unit 130, as depicted particularly in FIGS. 2A and 2B, can be configured to receive a hand and/or a forearm through an opening defined in the unit 130. Further description of stimulation delivery unit 130 is provided in a subsequent section.

Control module 110 can include electric circuitry and processing equipment as needed to control operation of peripheral sensory stimulation system 100 and to receive electricity from power source 120. Control module 110 may include an integrated air compressor for providing compressed air through tubing 125 and into stimulation delivery unit 130. The integrated air compressor may be sized as appropriate to fit within the geometric form factor provided by the control module 110. In an embodiment, the air compressor may be located external to the control module 110 rather than being integrated. In an embodiment, control module 110 may include a thermocouple and a thermal regulator to control the temperature of compressed air therein prior to the air being delivered to the stimulation delivery unit 130.

Control module 110 is illustrated as having a cubical geometry with a housing including a plurality of sides 112, though other geometries such as spherical, cylindrical, or other suitable geometries are contemplated by the present disclosure. In an embodiment, control module 110 may include a pressure regulator knob for adjusting air pressure (i.e., increasing or decreasing the degree of stimulation) and a switch 115 for alternating between continuous stimulation therapy and synchronized breathing therapy. Handle 114, tubing coupling 116, and power source coupling 118 may be positioned on various suitable locations of control module 110, and may be sized and shaped in various manners known to one of ordinary skill in the art.

Power source 120 is generally configured to provide electrical power to control module 110, which in turn provides compressed air and control operations to stimulation delivery unit 130. Power source 120 may comprise any suitable power device known to one of ordinary skill in the art (e.g., in-home wall outlet with 120 Volts, external power device, internal power device inside control module 110). In embodiments, power source 120 may include an electrical cable couplable to power source coupling 118 of control module 110. Power source 120 can be configured to transmit alternating current (AC) power, direct current (DC) power, or a combination of AC and DC power.

Tubing 125 can be used to enable the transfer of compressed air from control module 110 to stimulation delivery unit 130 for subsequent stimulation therapy. The transfer of compressed air may be based on one or more user or medical practitioner-inputted settings for control module 110, or may be automatic based on pre-programed settings for optimal stimulation therapy. Tubing 125 may extend from tubing coupling 116 of control module 110, to a corresponding tubing coupling 134 of stimulation delivery unit 130. Tubing 125 generally has a diameter between 0.1 and 2 inches and a length between 5 and 20 inches, though other diameter and length measurements less than or greater than these ranges are contemplated by the present disclosure. Tubing 125 can be made from a variety of materials including metals (e.g., aluminum), polymers (e.g., polyvinyl chloride), and composites, though other suitable materials known to one of ordinary skill in the art are contemplated by the present disclosure.

Stimulation delivery unit 130 may include a housing 131 having a plurality of sides 132 connected to form a geometric form factor (e.g., rectangular, spherical, cylindrical, triangular, computer mouse-shaped, etc.), a tubing coupling 134 configured to receive a second end of tubing 125 opposite the first end of tubing 125 received by tubing coupling 116, a replaceable therapy pad 136 positioned against a floor surface of unit 130 such that a user can rest their hand and/or forearm against the pad 136 during stimulation therapy, and a plurality of apertures 138 defined in a top side 132 through which a plurality of air tubes 142 (i.e., tubing and tubing connector) extend into. Stimulation delivery unit 130 may be manufactured from polymers (e.g., medical grade polymers), metals, composites, or combinations thereof, though other suitable materials are contemplated by the present disclosure.

In embodiments, the geometric form factor provided by housing 131 and plurality of sides 132 can be configured to receive a hand and/or a forearm of a therapy user, as illustrated particularly in FIGS. 2A and 2B. In embodiments, one or more of the plurality of sides 132 may define at least one opening as illustrated particularly in FIGS. 1A and 1B. Replaceable therapy pad 136 can be made from any suitable foam, gel, or soft material known to one of ordinary skill in the art.

Plurality of apertures 138 may be dispersed in a pattern or irregularly along one or more of the plurality of sides 132. For example, plurality of apertures 138 may have a defined pattern that enables optimal dispersion of compressed air into stimulation delivery unit 130 (e.g., a pattern that matches the structure of a user forearm as illustrated in FIG. 2B, for example), or apertures 138 may be dispersed randomly, or may have a combination of both aperture dispersal configurations. Plurality of apertures 138 should have a diameter suitable to receive a single air tube 142 of plurality of air tubes 142 into each aperture 138 (e.g., aperture diameter is generally less than one inch). In embodiments, plurality of apertures 138 can be machined into housing 131 after manufacturing, or can be designed into housing 131 such that apertures 138 are formed during manufacturing of housing 131 rather than being subsequently machined in (e.g., when housing 131 is molded).

Plurality of air tubes 142 can be couplable to tubing 125 such that compressed air from a compressor is evenly transferred throughout air tubes 142 which are partially located within apertures 138. For example, each air tube 142 may include a tubing portion and a connector portion, as illustrated particularly in FIGS. 1B and 2A. The tubing portion may be located external to housing 131, while the connector portion can be received within an aperture 138. Compressed air can flow from the tubing portion into the connector portion and lastly into the housing 131 for use during stimulation therapy. In embodiments, plurality of air tubes 142 can include five to twenty individual tubes 142, or a number of tubes less than five or greater than twenty if suitable compressed air can be supplied to the inside of housing 131 (e.g., compressor output can be increased to enable use with less air tubes 142, for example).

Plurality of air tubes 142 may be positioned such that puffs of air are directed to the volar surface of a forearm, a palm of a hand, and/or the fingers of a user. In embodiments, peripheral sensory stimulation is provided to the hand, via the plurality of air tubes 142, through puffs of air to encourage collateral blood supply and sensorimotor development in the user. Improvements in neurological disorder symptoms are achieved through use of peripheral sensory stimulation system 100 with directed puffs of air and/or synchronized breathing techniques.

In general, peripheral sensory stimulation system 100 is designed to accelerate healing and recovery from various neurological disorders including, but not limited to, stroke, traumatic brain injury, PTSD, Parkinson's disease, anxiety, depression, insomnia, etc. Peripheral sensory stimulation system 100 can achieve the acceleration in healing and recovery by providing low levels of physical stimulation, via the stimulation delivery unit 130, to the hand and/or forearm of a user in a linear or random manner throughout a therapy session (e.g., bursts of air or continuous air). Physical stimulation can encourage the brain to strengthen existing neurological pathways or to build new pathways in response to the stimulation.

Peripheral sensory stimulation system 100 can be self-contained and powered from a standard wall outlet in a clinical setting or a home setting. Peripheral sensory stimulation system 100 can be used with a user that is sitting or lying down. The user can use the stimulation delivery unit 130 in a “palm up” position (i.e., the palm of the hand facing upward), and the user can use either their right or left hand as preferred or as instructed by a medical practitioner or other therapy supervisor.

In operation, a user can position peripheral sensory stimulation system 100 on a flat surface such as a countertop, the ground, or a table as illustrated in FIG. 1B. The user can maneuver the control module 110 using the provided handle 114 to the flat surface. The power source 120 and the stimulation delivery unit 130 can be maneuvered to the flat surface in any suitable manner. The user or another person such as a medical practitioner should ensure that the electrical cable is connected to both the control module 110 and the power source 120 to ensure proper delivery of electricity. The stimulation delivery unit 130 should be prepared for use by ensuring that tubing 125 and the plurality of air tubes 142 are untangled and clear of any obstructions. Generally, the user will want to record the time and date of the stimulation therapy session in a logbook. The user or other person can turn on the power source 120 using a switch or button located on a side 112 of the control module 110 near the power source coupling 118. The integrated air compressor inside the control module 110 will activate in response to activation of the power source 120, but this does not immediately start therapy with stimulation delivery unit 130.

In an embodiment, control module 110 may include a light-emitting diode (LED) 113 configured to emit continuous flashes of light to indicate therapy duration and to help synchronize user breathing when using pulsed therapy (pulsed therapy being cycled bursts of air rather than a continuous burst of air during continuous therapy; control module 110 may have a switch 115 for alternating between pulsed therapy and continuous therapy). For example, the LED 113 may emit twelve rapid light flashes to indicate a therapy time of thirty minutes, or five slow light flashes to indicate a therapy time of forty-five minutes. The therapy time may be adjusted by the user or other person as needed to achieve various treatment outcomes for that user (the LED 113 light flash configuration could change with an update to therapy time). The flashes of light can also be used as a measure of when control module 110 is cycled on and cycled off, so that a user may synchronize their breathing to match the cycled-on times.

After setting the therapy time, the user may place their hand and/or forearm into the stimulation delivery unit 130 with the palm of the hand facing upward, and the back of the hand and forearm optionally resting against replaceable therapy pad 136 (to provide a comfortable resting surface for the duration of therapy). The user should verify that their hand and/or forearm are properly inserted into the stimulation delivery unit 130 (arranged within housing 131 such that the hand and/or forearm are oriented below plurality of apertures 138 and air tubes 142) before commencing stimulation therapy which can be activated via the control module 110.

Activation of stimulation therapy can enable the delivery of intermittent puffs of compressed air from control module 110 through tubing 125 and plurality of air tubes 142 and into housing 131 at locations along the user's hand and/or forearm, as illustrated particularly in FIGS. 2A and 2B. In an embodiment, the intermittent puffs of air can cycle between one second on and two seconds off, for example, to provide a stream of air puffs to the user. Alternative air cycling configurations may be used, such as five seconds on and five seconds off, or other suitable on/off cycling relationships. The distribution of air puffs against a user's hand and/or forearm facilitates the rehabilitation, improvement, or restoration of motion in users with neurological disorders, and also facilitates treatment of users in comatose and in natal settings, for example.

In an embodiment, the delivery of intermittent puffs of air can be characterized as “pulsed” in that random stimulation via pulsed air bursts occurs for approximately five seconds, for example (random stimulation meaning that not all puffs of air are delivered through stimulation delivery unit 130 at the same time). During the five-second cycle (or other suitable cycle configuration), the user may inhale while observing LED 113, and when LED 113 turns off, the user may exhale for approximately five seconds to match the duration of air bursts. This process synchronizes the user's breathing with the air cycling configuration used by peripheral sensory stimulation system 100. In an embodiment, the delivery of intermittent puffs of air can be characterized as “continuous” with the delivery of puffs of air occurring continuously. With this embodiment, there is not cycling on and off as with “pulsed” delivery-instead, the user may breathe as they desire while continuous puffs or bursts of air are delivered to the appendage(s) applied to peripheral sensory stimulation system 100. “Continuous” delivery may also be random in that not all puffs or bursts of air are delivered through stimulation delivery unit 130 at the same time.

Stimulation therapy with peripheral sensory stimulation system 100 may include one hour of therapy per day (e.g., two thirty-minute sessions, one session for each hand) for a total of four weeks. During a therapy session, users may also synchronize their breathing to the air cycling configuration of system 100. For example, when the cycle begins a five second round of stimulation via air dispersal, the user will be asked to take a deep breath and hold their breath until the cycle ends, at which point the user can exhale fully until the next cycle begins at the end of a five second off period (or other on/off air cycling time relationships). In general, users who complete less than forty-five minutes of treatment per day, for example, will be considered as not receiving adequate treatment and may be instructed to alter their treatment regimen to compensate. Alternatively, one user may react differently to shorter stimulation therapy than another user and therefore may still be compliant even though their total treatment time per day is less than forty-five minutes or another prescribed time.

In an embodiment where peripheral sensory stimulation system 100 is used in a medical setting, for example with magnetic resonance imaging (MRI), control module 110 may be positioned outside of the MRI operating room. This may require tubing 125 to be routed from outside of the room to the inside where the stimulation delivery unit 130 is located. Accordingly, in such an embodiment, tubing 125 may be longer than normal to accommodate the increased distance between the control module 110 and the stimulation delivery unit 130.

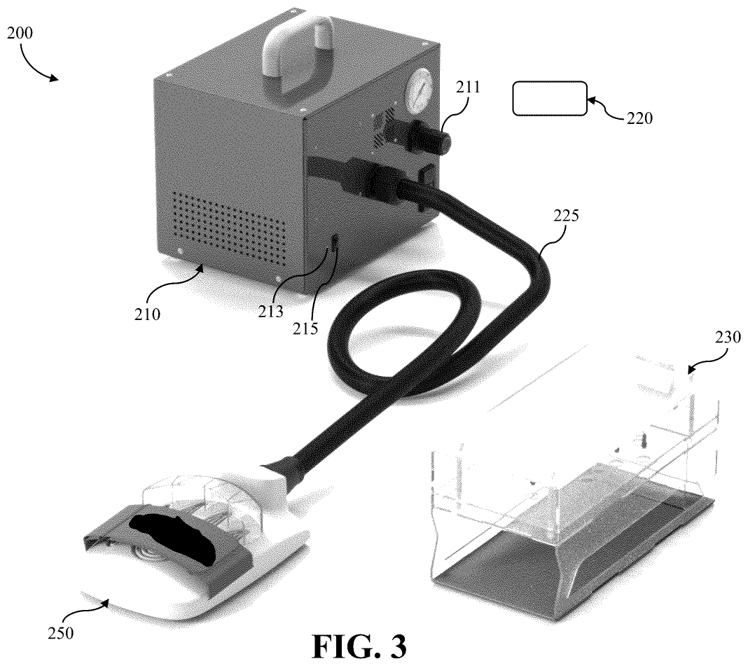

Referring now generally to FIG. 3, a peripheral sensory stimulation system 200 according to an embodiment of the present disclosure may include a control module 210 having an integrated air compressor provided within a housing, a handle on a top side of the control module 210, a tubing coupling connectable to a side of the control module 210, and a power source coupling defined within a side of the control module 210. The peripheral sensory stimulation system 200 may further include a power source 220, tubing 225, a first stimulation delivery unit 230 couplable to the control module 210 via the tubing 225, and a second stimulation delivery unit 250 couplable to the control module 210 via the tubing 225. In an embodiment, control module 210 may have a pressure regulator knob 211 configured to adjust air pressure (i.e., increasing or decreasing the degree of stimulation), and a switch 215 for alternating between continuous stimulation therapy (i.e., “continuous” delivery of air) and synchronized breathing therapy (i.e., “pulsed” delivery of air). In an embodiment, control module 210 may include a thermocouple and a thermal regulator to control the temperature of air therein prior to the air being delivered to the first and/or second stimulation delivery units 230, 250. In an embodiment, control module 210 may include an LED 213 configured to operate identical to LED 113 of control module 110.

First stimulation delivery unit 230 can be configured to receive a hand and/or a forearm through an opening defined in the unit 230. Second stimulation delivery unit 250 will be described in further detail in a subsequent section. In an embodiment, first stimulation delivery unit 230 is configured for palm-up chamber stimulation delivery, and second stimulation delivery unit 250 is configured for palm-down stimulation delivery when positioned on a flat surface. Alternatively, second stimulation delivery unit 250 can be positioned upside down such that a bottom surface of unit 250 and the user's palm are both facing upward.

Peripheral sensory stimulation system 200 has many similarities to peripheral sensory stimulation system 100, and for simplicity the description of common components is not repeated in the following. Like reference numerals and features names may designate like feature names throughout that are corresponding or analogous. Moreover, it should be understood that peripheral sensory stimulation system 200 can be configured to operate identical or similar to system 100, with the addition of second stimulation delivery unit 250 to system 200. Second stimulation delivery unit 250 can be configured to operate in a similar manner as first stimulation delivery unit 250 and stimulation delivery unit 150.

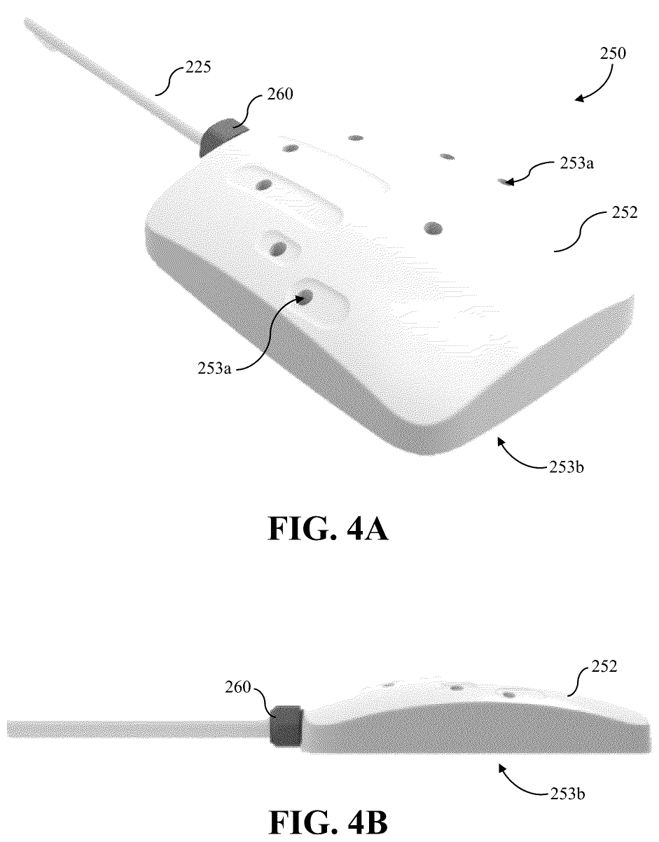

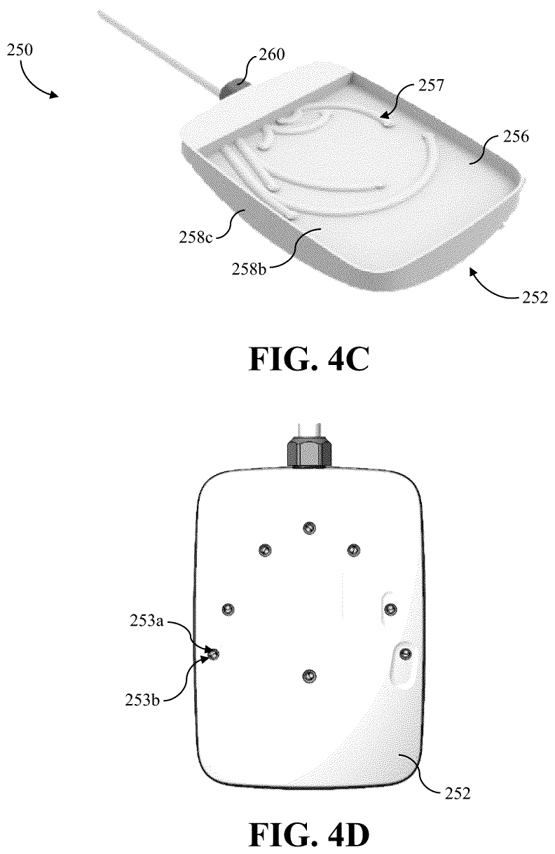

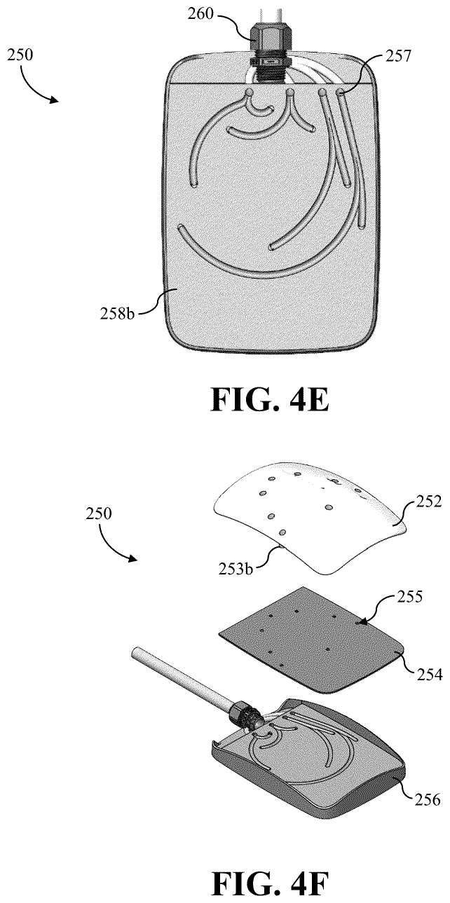

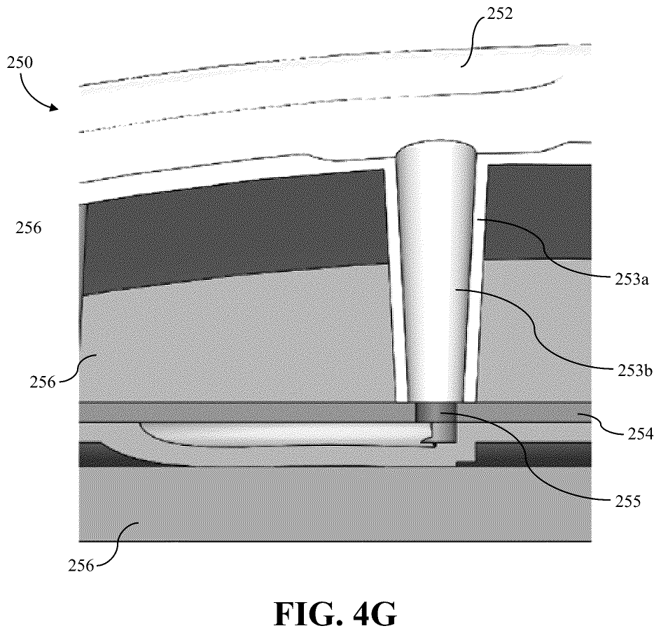

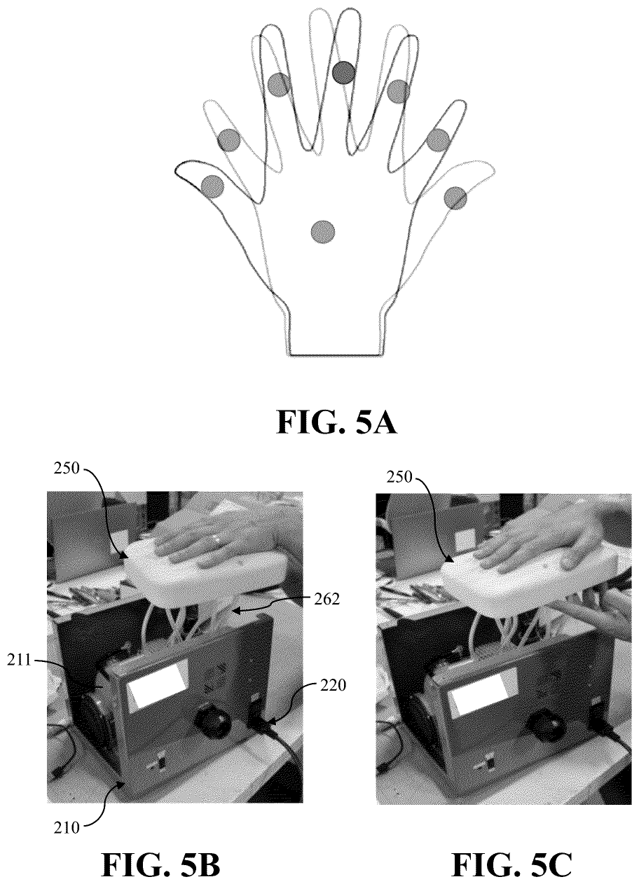

Referring now generally to FIGS. 4A-5C, second stimulation delivery unit 250 may include an upper housing 252 having a generally convex surface, a plurality of apertures 253a defined in the upper housing 252 through which a plurality of tubing connectors 253b may partially extend, a gasket 254 having a plurality of apertures 255 aligned with the plurality of apertures 253a of upper housing 252, and a lower housing 256 defining a plurality of channels 257, the lower housing 256 being couplable to the upper housing 252. The lower housing 256 may have a top surface 258a separated from a bottom surface 258b by a perimeter edge 258c to form the overall structure of lower housing 256. The second stimulation delivery unit 250 may further include a tubing coupling 260 configured to receive tubing, such as tubing 225, and a plurality of air tubes 262 for delivering compressed air through unit 250 as illustrated particularly in FIGS. 5B and 5C.

Second stimulation delivery unit 250 can be designed to resemble a computer mouse for ergonomic hand palm-down use during stimulation therapy. Upper housing 252 of second stimulation delivery unit 250 may have a generally convex surface to mimic the generally convex shape of a hand palm. In an embodiment, upper housing 252 may have a generally flat surface. Upper housing 252 may define a plurality of apertures 253a dispersed in a circular configuration to match the shape of a left hand or a right hand. For example, as illustrated particularly in FIGS. 5A-5C, upper housing 252 can define separate apertures 253a for receiving each finger of a left hand and a right hand, and a centrally located aperture 253a to be received in the center of either the left hand or the right hand. This enables second stimulation delivery unit 250 to be used with either the left hand as shown in FIG. 5B, or the right hand as shown in FIG. 5C, depending on various factors including user preference, feasibility, and user comfort, for example. In an embodiment, a plurality of slots may be defined in upper housing 252 at the locations of the plurality of apertures 253a to match the longitudinal orientation of fingers against upper housing 252. This can further enhance ergonomic use for the user of second stimulation delivery unit 250.

Plurality of tubing connectors 253b may extend from plurality of apertures 253a through a portion of lower housing 256 and up to the plurality of apertures 255 defined by gasket 254. Plurality of tubing connectors 253b facilitate the transfer of air through lower housing 256 and gasket 254 and into plurality of apertures 253a of upper housing 252 during stimulation therapy. Accordingly, air is transferred to the fingers, palm, and/or forearm of a user when they are using second stimulation delivery unit 250. Gasket 254 is generally a thin piece of material that provides a barrier between upper and lower housings 252, 254. In an embodiment, gasket 254 may have a generally rectangular geometry with one or more rounded edges. Gasket 254 can define a plurality of apertures 255 dispersed to match the pattern of plurality of apertures 253a of upper housing 252. The interface between upper housing 252, gasket 254, and lower housing 256 is illustrated particularly in the cross-sectional view of FIG. 4G.

Lower housing 256 may also have a generally convex shape to match the shape of upper housing 252. Lower housing 256 may define a plurality of channels 257, some of which may be interconnected. Plurality of channels 257 are generally spaced to match the pattern of plurality of apertures 253a to enable transfer of air throughout lower housing 256. Plurality of channels 257 may have an arc design as illustrated in FIG. 4E, or may be generally linear without an arc. In an embodiment, lower housing 256 may include a top surface 258a separated from a bottom surface 258b by a perimeter edge 258c as illustrated in FIGS. 4E and 4F. The perimeter edge 258c generally extends from the bottom surface 258b to a height above the top surface 258a such that a lip surrounding the top surface 258a is created. The perimeter edge 258c may have rounded corners and may vary in height at different locations (e.g., as illustrated, perimeter edge 258c may have a sloping edge on each side).

Second stimulation delivery unit 250 may further include a tubing coupling 260 configured to receive tubing such as tubing 225, and a plurality of air tubes 262 for receiving air through upper and lower housings 252, 256 and gasket 254. Tubing coupling 260 may be positioned on a frontmost surface of unit 250 or in any other suitable location where tubing 225 can be received. Plurality of air tubes 262 are generally positioned near bottom surface 258b of lower housing 256 to enable delivery of air from lower housing 256 to plurality of apertures 253a defined in upper housing 252 where a user's body part (e.g., fingers, hand, forearm) is generally positioned.

In operation, a user may assemble peripheral sensory stimulation system 200 in a similar manner as peripheral sensory stimulation system 100, but with the addition of second stimulation delivery unit 250. System 200 may be configured such that first and second stimulation delivery units 230, 250 may be used simultaneously or individually depending on various factors such as user requirements and preference. In an embodiment, separate users may receive simultaneous stimulation therapy from first and second stimulation delivery units 230, 250 during a single therapy session (i.e., control module 210 can have multiple connection points for tubing 225). Therapy session and air puff cycling times for system 200 are generally the same as those for system 100.

To use second stimulation delivery unit 250, a user may place either their left hand or their right hand on the upper housing 252 such that the user's fingers, palm of the hand, and/or forearm aligns with the plurality of apertures 253a. The user may grasp second stimulation delivery unit 250 as they would a computer mouse or in any other manner that provides the most comfort (e.g., the user may keep their fingers straight as stimulation therapy is delivered as illustrated in FIGS. 5B and 5C). Control module 210 and the integrated compressor may be turned on as power is provided by power source 220. Compressed air can be delivered through tubing 225 and into second stimulation delivery unit 250 for use in stimulation therapy. The user may synchronize their breathing with the dispersion of air puffs in a similar manner as they would for stimulation delivery unit 130 of system 100. In embodiments, delivery of air can be “pulsed” or “continuous” as those terms are used with respect to stimulation delivery unit 130.

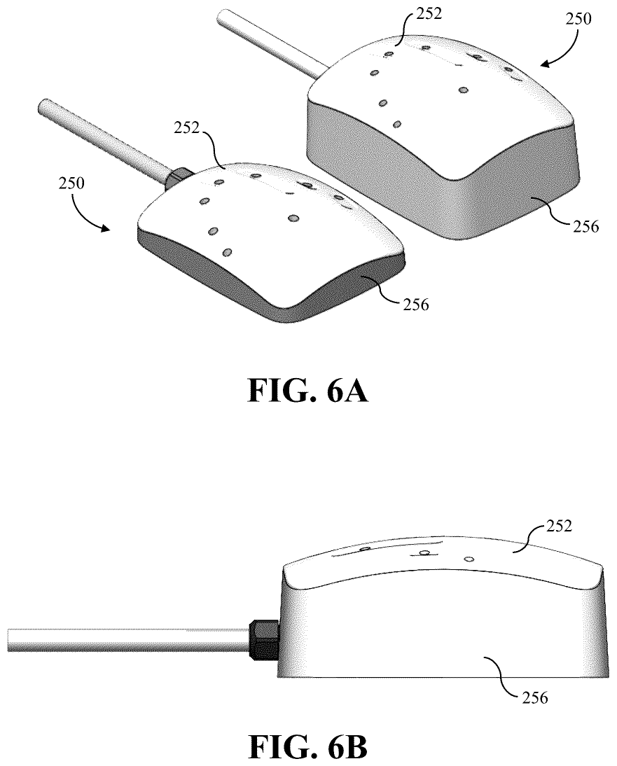

Referring now generally to FIGS. 6A and 6B, the height of lower housing 256 may vary depending on the preference of the user, the requirements for therapy, or various other factors, according to an embodiment of the present disclosure. For example, the height of lower housing 256 may be less than two inches in an embodiment or may be greater than two inches in another embodiment, both of which are illustrated in FIG. 6A. The height of upper housing 252 may vary to match or compensate for a change in height of lower housing 256. The specific location of tubing coupling 260 on lower housing 256 may also vary in response to height changes (e.g., tubing coupling 260 may be positioned higher or lower with respect to the height of lower housing 256).

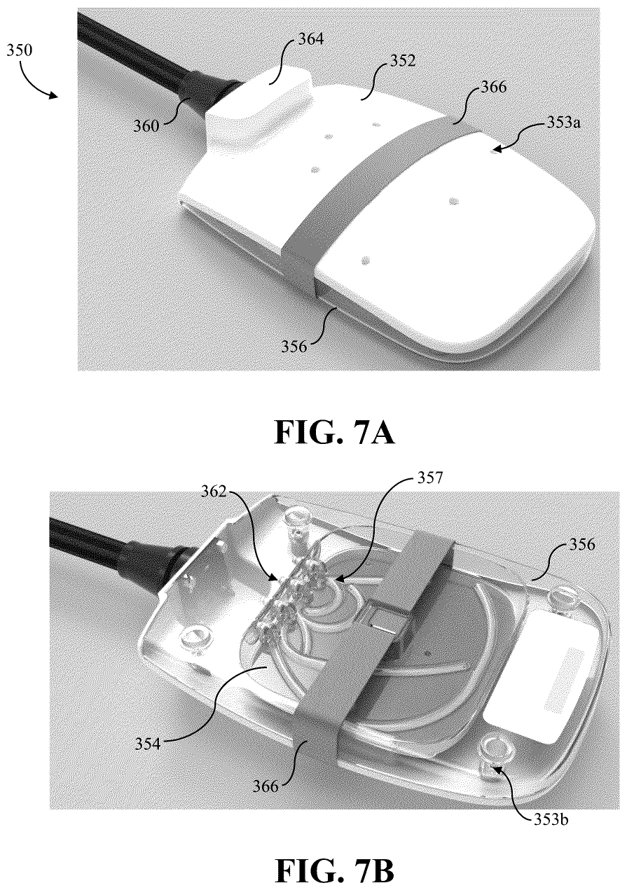

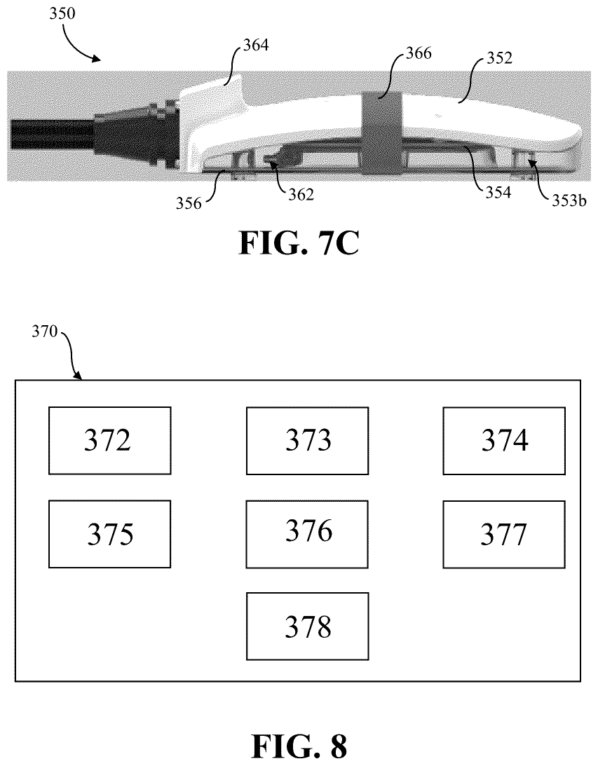

Referring now generally to FIGS. 7A-8, a stimulation delivery unit 350 according to an embodiment of the present disclosure may include an upper housing 352 having a generally convex surface, a plurality of apertures 353a defined in the upper housing 352 through which a plurality of tubing connectors 353b may partially extend, a gasket 354 in contact with the upper housing 352, and a lower housing 356 defining a plurality of channels 357, the lower housing 356 being couplable to the upper housing 352. In an embodiment, the lower housing 356 may be transparent and may include one or more feet extending from a bottom portion, such that the stimulation delivery unit 350 can be positioned with the one or more feet against a surface for stability. The stimulation delivery unit 350 may further include a tubing coupling 360 configured to receive tubing, and a plurality of air tube connectors 362 for receiving air. In an embodiment, housing 352 may have a generally flat surface instead of a generally convex surface.

Stimulation delivery unit 350 has many similarities to stimulation delivery unit 250, and for simplicity the description of common components is not repeated in the following. Like reference numerals and features names may designate like feature names throughout that are corresponding or analogous. Moreover, it should be understood that stimulation delivery unit 350 can be configured to operate identical or similar to unit 250. Instructions or suggested steps for use of unit 250 for stimulation therapy can be applied exactly or similarly to unit 350 for use in stimulation therapy. Additional features and/or differences between unit 350 and unit 250 will now be described. Figure comparison highlights the referenced additional features and/or differences along with other features and/or differences not explicitly described herein.

The stimulation delivery unit 350 may further include a connector ridge 364 integral with a top surface of the upper housing 352. The connector ridge 364 may be configured to receive tubing coupling 360 and to facilitate the delivery of air from tubing into unit 350 for use with stimulation therapy. The stimulation delivery unit 350 may further include a band 366 secured around a mid-portion of the upper and lower housings 352, 356. Band 366 may be used to secure a user's hand to the stimulation delivery unit during stimulation therapy (i.e., the user places their hand under the band which can be tightened to restrict hand movement during therapy). Additionally, band 366 may be removed in order to gain access to the inside of lower housing 356 which includes, among other things, gasket 354 and plurality of channels 357.

As illustrated in FIG. 8, stimulation delivery unit 350 may further include a variety of internal housing components 370, both of a mechanical and an electrical nature. Housing components 370 may include a plurality of solenoids 372 for converting electrical energy into mechanical work, a programming module 373 for inputting and outputting therapy settings, a regulator 374 to ensure a steady supply of electricity into unit 350, a cooling fan 375 to cool the housing components 370 which thereby cools upper and lower housings 352, 356, a voltage converter 376 to convert between voltage types or amounts, a power entry module 377 integrated with other electrical components such as a switch and a circuit breaker, and an optional integrated air compressor 378. Alternatively, one or more housing components 370 may be located external to stimulation delivery unit 350, but in proximity to unit 350 to be operationally usable. Additional housing components 370 known to one of ordinary skill may be included as needed.

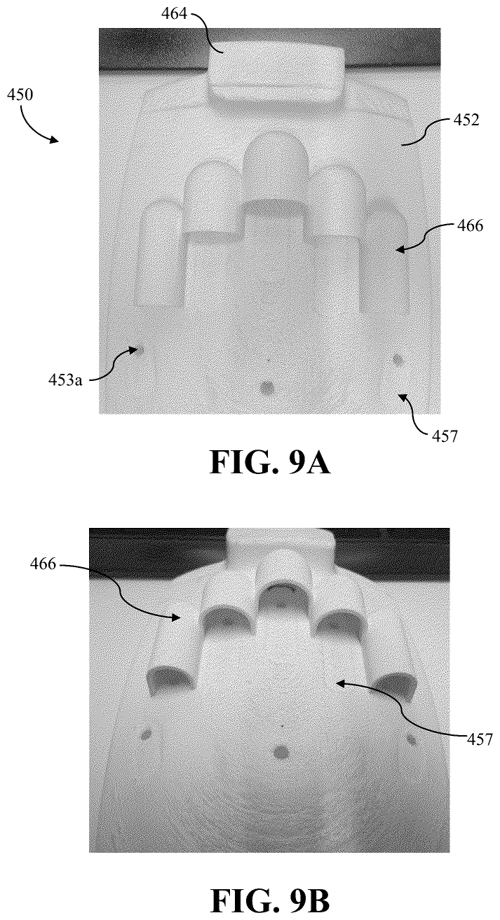

Referring now generally to FIGS. 9A and 9B, a stimulation delivery unit 450 according to an embodiment of the present disclosure may include a housing 452, a plurality of apertures 453a defined in the housing 452, a plurality of channels 457 defined in the housing, the channels 457 being defined in the same general positions as the apertures 453a, a connector ridge 464 integral with a top surface of the housing 452 and configured to receive a tubing coupling, and a tunnel structure 466 configured to receive the little, ring, middle, and index fingers of a user's left or right hand. Tunnel structure 466 is generally integral with housing 452 as illustrated in the figures. In embodiments, tunnel structure 466 is open from a first end to a second end, or is open at a first end and closed at a second end. Tunnel structure 466 may include one or more dividers separating the individual finger slots.

Stimulation delivery unit 450 has many similarities to stimulation delivery units 250, 350, and for simplicity the description of common components is not repeated in the following. Like reference numerals and features names may designate like feature names throughout that are corresponding or analogous. Moreover, it should be understood that stimulation delivery unit 450 can be configured to operate identical or similar to units 250, 350. Instructions or suggested steps for use of units 250, 350 for stimulation therapy can be applied exactly or similarly to unit 450 for use in stimulation therapy. Comparison of figures will highlight various differences and/or feature additions between units 250, 350, and 450. Such differences and/or feature additions are not explicitly described herein, though it should be understood that the individual features of units 250, 350, and 450 may be combined in any suitable manner to achieve optimal therapy.

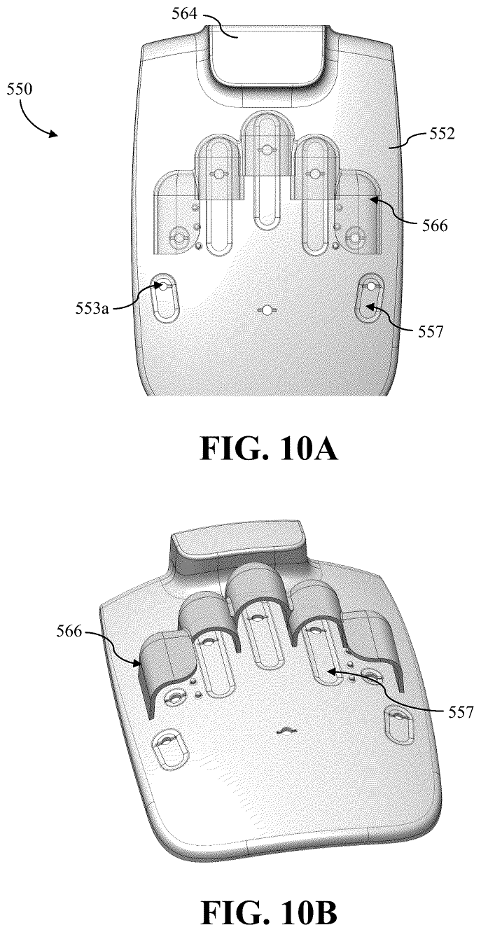

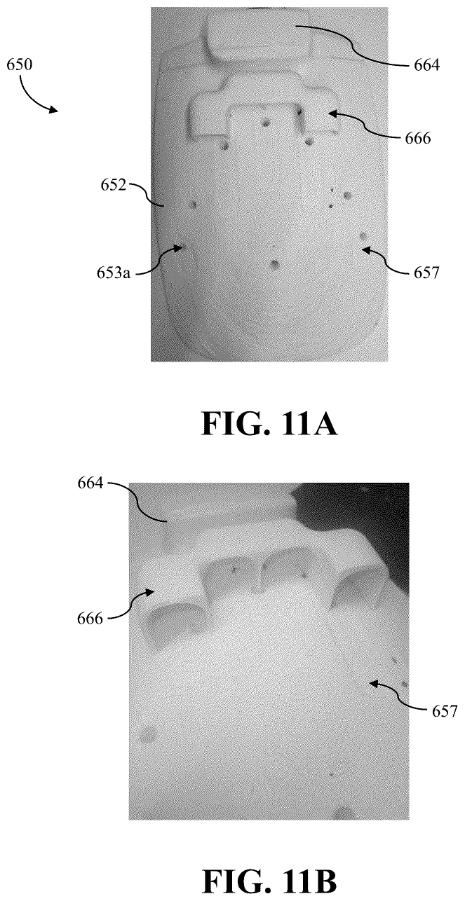

Referring now generally to FIGS. 10A and 10B, a stimulation delivery unit 550 according to an embodiment of the present disclosure may include a housing 552, a plurality of apertures 553a defined in the housing 552 through which a plurality of tubing connectors may partially extend, a plurality of channels 557 defined in the housing, the channels 557 being defined in the same general positions as the apertures 553a, a connector ridge 564 integral with a top surface of the housing 552 and configured to receive a tubing coupling, and a tunnel structure 566 configured to receive the little, ring, middle, and index fingers of a user's left or right hand. Tunnel structure 566 is generally integral with housing 552 as illustrated in the figures. In embodiments, tunnel structure 566 is open from a first end to a second end, or is open at a first end and closed at a second end. Tunnel structure 566 may include one or more dividers separating the individual finger slots.

Stimulation delivery unit 550 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 550 can be configured to operate identical or similar to previously described units. Instructions or suggested steps for use of previously described units for stimulation therapy can be applied exactly or similarly to unit 550 for use in stimulation therapy. Comparison of figures will highlight various differences and/or feature additions between the previously described units and unit 550. Such differences and/or feature additions are not explicitly described herein, though it should be understood that the individual features of the previously described units and unit 550 may be combined in any suitable manner suitable to achieve optimal therapy.

Referring now generally to FIGS. 11A and 11B, a stimulation delivery unit 650 according to an embodiment of the present disclosure may include a housing 652, a plurality of apertures 653a defined in the housing 652 through which a plurality of tubing connectors may partially extend, a plurality of channels 657 defined in the housing, the channels 657 being defined in the same general positions as the apertures 653a, a connector ridge 664 integral with a top surface of the housing 652 and configured to receive a tubing coupling, and a tunnel structure 666 configured to receive the little, ring, middle, and index fingers of a user's left or right hand. Tunnel structure 666 is generally integral with housing 652 as illustrated in the figures. In embodiments, tunnel structure 666 is open from a first end to a second end, or is open at a first end and closed at a second end. Tunnel structure 666 may include one or more dividers separating the individual finger slots.

Stimulation delivery unit 650 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 650 can be configured to operate identical or similar to previously described units. Instructions or suggested steps for use of previously described units for stimulation therapy can be applied exactly or similarly to unit 650 for use in stimulation therapy. Comparison of figures will highlight various differences and/or feature additions between the previously described units and unit 650. Such differences and/or feature additions are not explicitly described herein, though it should be understood that the individual features of the previously described units and unit 650 may be combined in any suitable manner suitable to achieve optimal therapy.

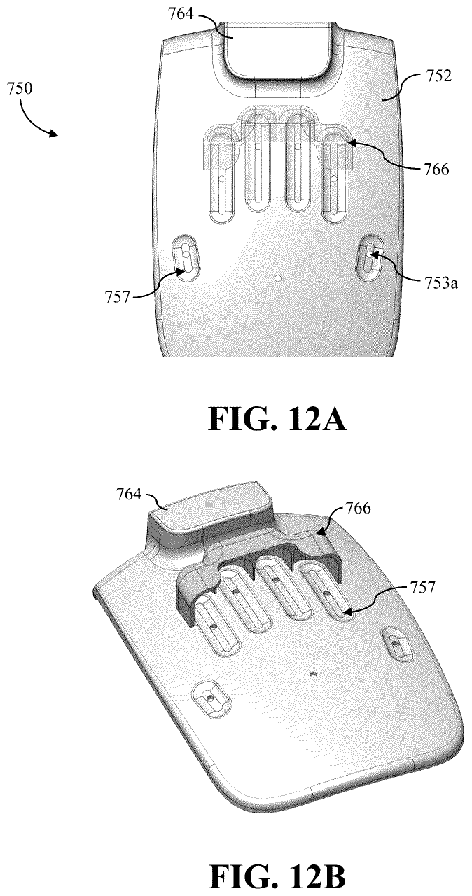

Referring now generally to FIGS. 12A and 12B, a stimulation delivery unit 750 according to an embodiment of the present disclosure may include a housing 752, a plurality of apertures 753a defined in the housing 752 through which a plurality of tubing connectors may partially extend, a plurality of channels 757 defined in the housing, the channels 757 being defined in the same general positions as the apertures 753a, a connector ridge 764 integral with a top surface of the housing 752 and configured to receive a tubing coupling, and a tunnel structure 766 configured to receive the little, ring, middle, and index fingers of a user's left or right hand. Tunnel structure 766 is generally integral with housing 752 as illustrated in the figures. In embodiments, tunnel structure 766 is open from a first end to a second end, or is open at a first end and closed at a second end. Tunnel structure 766 may include one or more dividers separating the individual finger slots.

Stimulation delivery unit 750 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 750 can be configured to operate identical or similar to previously described units. Instructions or suggested steps for use of previously described units for stimulation therapy can be applied exactly or similarly to unit 750 for use in stimulation therapy. Comparison of figures will highlight various differences and/or feature additions between the previously described units and unit 750. Such differences and/or feature additions are not explicitly described herein, though it should be understood that the individual features of the previously described units and unit 750 may be combined in any suitable manner suitable to achieve optimal therapy.

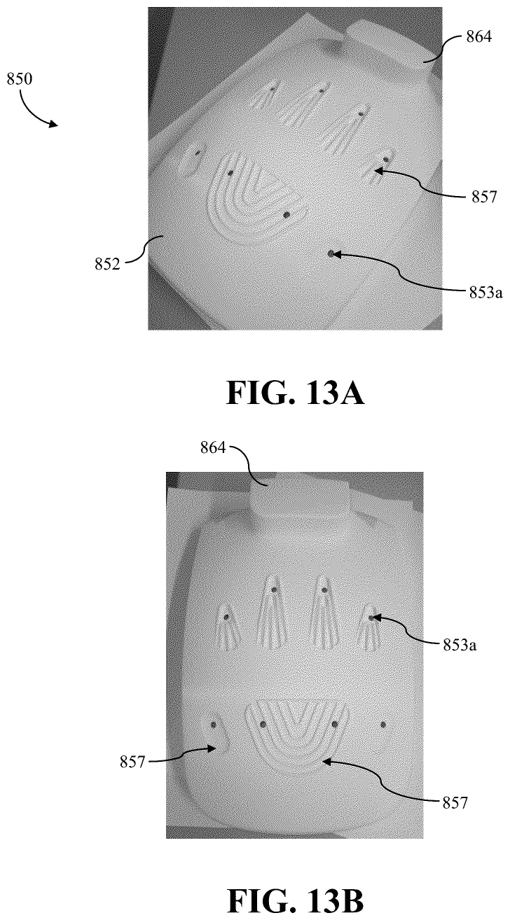

Referring now generally to FIGS. 13A and 13B, a stimulation delivery unit 850 according to an embodiment of the present disclosure may include a housing 852, a plurality of apertures 853a defined in the housing 852 through which a plurality of tubing connectors may partially extend, a plurality of channels 857 (e.g., ribs) defined in the housing, the channels 857 being defined in the same general positions as the apertures 853a, and a connector ridge 864 integral with a top surface of the housing 852 and configured to receive a tubing coupling. Like previous stimulation delivery unit embodiments, unit 850 is configured to receive a hand and/or forearm of a user against the top surface of the housing 852. For example, a user may place their hand against the top surface such that the hand palm and the thumb are in contact with the plurality of apertures 853a and the channels 857 located distal to the connector ridge 864. The remaining fingers may be placed in contact with the plurality of apertures 853a and channels 857 located proximal to the connector ridge 864. Air from a control module or another device may be directed through the plurality of apertures 853a to the hand palm and each finger for stimulation therapy.

Stimulation delivery unit 850 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 850 can be configured to operate identical or similar to previously described units. Instructions or suggested steps for use of previously described units for stimulation therapy can be applied exactly or similarly to unit 850 for use in stimulation therapy. Comparison of figures will highlight various differences and/or feature additions between the previously described units and unit 850. Such differences and/or feature additions are not explicitly described herein, though it should be understood that the individual features of the previously described units and unit 850 may be combined in any suitable manner suitable to achieve optimal therapy.

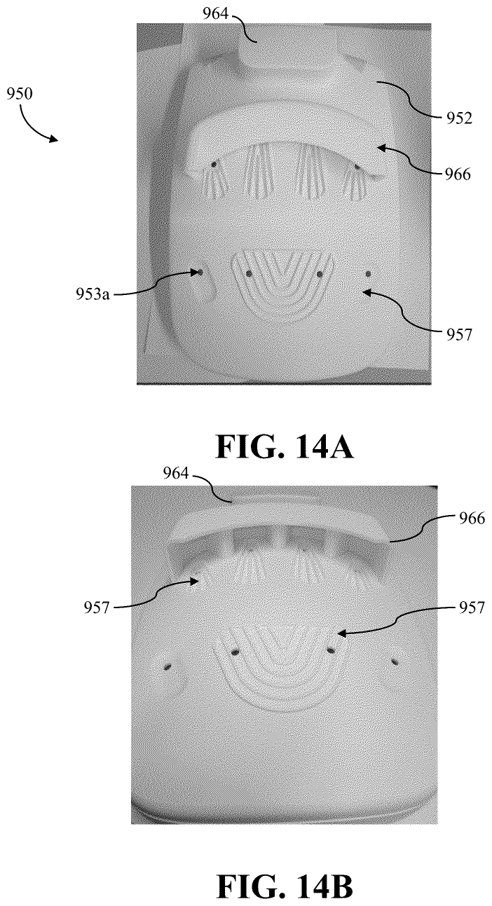

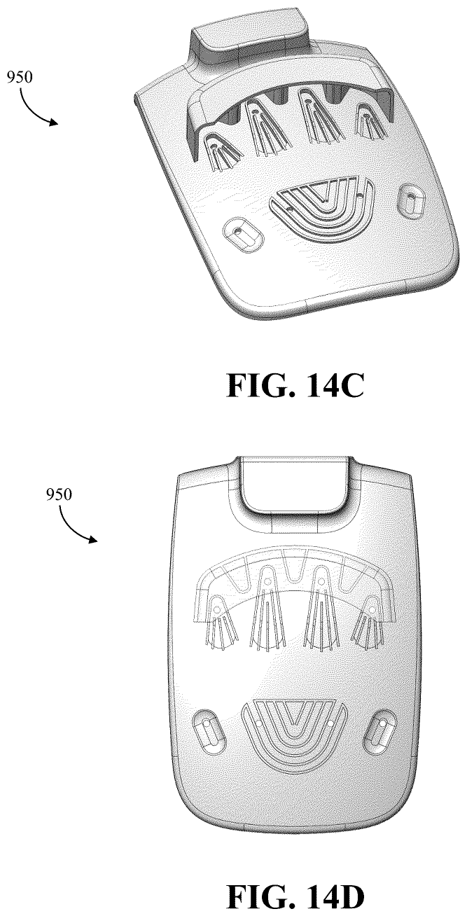

Referring now generally to FIGS. 14A-14D, a stimulation delivery unit 950 according to an embodiment of the present disclosure may include a housing 952, a plurality of apertures 953a defined in the housing 952 through which a plurality of tubing connectors may partially extend, a plurality of channels 957 (e.g., ribs) defined in the housing, the channels 957 being defined in the same general positions as the apertures 953a, a connector ridge 964 integral with a top surface of the housing 952 and configured to receive a tubing coupling, and a tunnel structure 966 configured to receive the little, ring, middle, and index fingers of a user's left or right hand. Tunnel structure 966 is generally integral with housing 952 as illustrated in the figures. In embodiments, tunnel structure 966 is open from a first end to a second end, or is open at a first end and closed at a second end. Tunnel structure 966 may include one or more dividers separating the individual finger slots.

Stimulation delivery unit 950 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 950 can be configured to operate identical or similar to previously described units. Instructions or suggested steps for use of previously described units for stimulation therapy can be applied exactly or similarly to unit 950 for use in stimulation therapy. Comparison of figures will highlight various differences and/or feature additions between the previously described units and unit 950. Such differences and/or feature additions are not explicitly described herein, though it should be understood that the individual features of the previously described units and unit 950 may be combined in any suitable manner suitable to achieve optimal therapy.

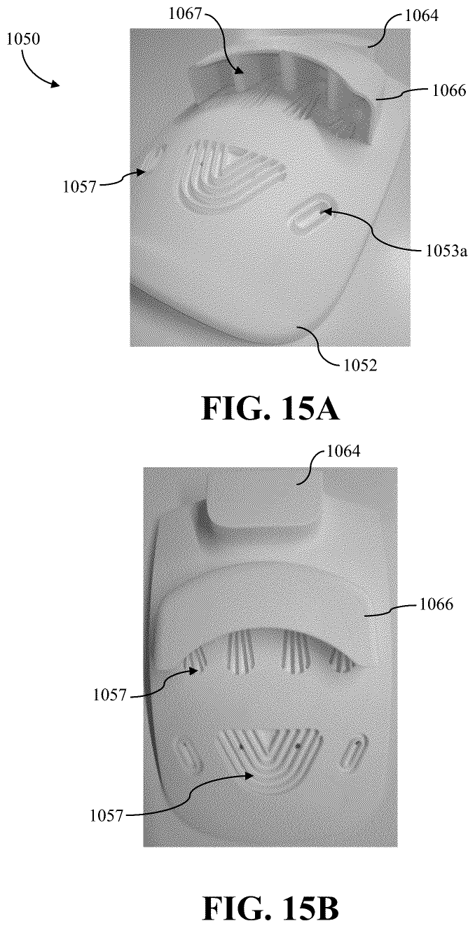

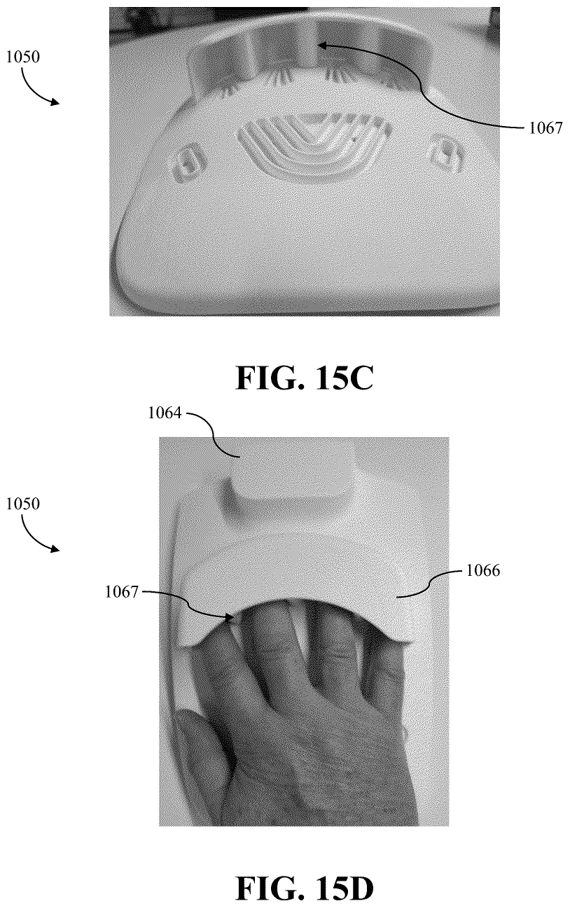

Referring now generally to FIGS. 15A-15D, a stimulation delivery unit 1050 according to an embodiment of the present disclosure may include a housing 1052, a plurality of apertures 1053a defined in the housing 1052 through which a plurality of tubing connectors may partially extend, a plurality of channels 1057 (e.g., ribs) defined in the housing, the channels 1057 being defined in the same positions as the apertures 1053a, a connector ridge 1064 integral with a top surface of the housing 1052 and configured to receive a tubing coupling, and a tunnel structure 1066 configured to receive the little, ring, middle, and index fingers of a user's left or right hand, as illustrated particularly in FIG. 15D. Tunnel structure 1066 is generally integral with housing 1052 as illustrated in the figures. In embodiments, tunnel structure 1066 is open from a first end to a second end, or is open at a first end and closed at a second end. Tunnel structure 1066 may include one or more dividers separating the individual finger slots.

Stimulation delivery unit 1050 has many similarities to previously described stimulation delivery units, and for simplicity the description of common components is not repeated in the following. Like reference numerals and features names may designate like feature names throughout that are corresponding or analogous. Moreover, it should be understood that stimulation delivery unit 1050 can be configured to operate identical or similar to previously described units. Instructions or suggested steps for use of previously described units for stimulation therapy can be applied exactly or similarly to unit 1050 for use in stimulation therapy. Comparison of figures will highlight various differences and/or feature additions between the previously described units and unit 1050. Such differences and/or feature additions are not explicitly described herein, though it should be understood that the individual features of the previously described units and unit 1050 may be combined in any suitable manner suitable to achieve optimal therapy.

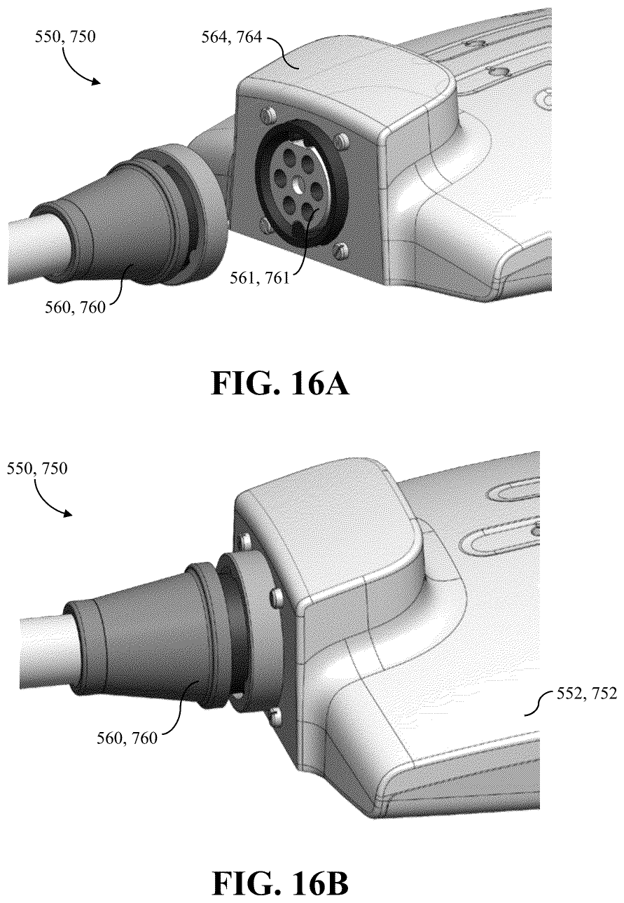

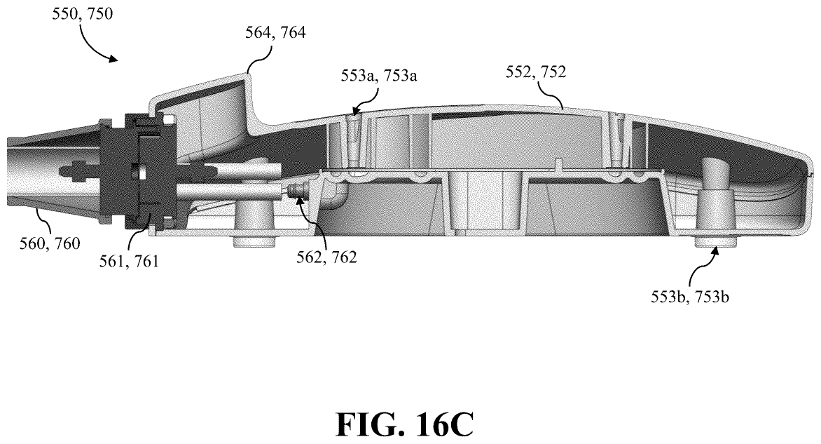

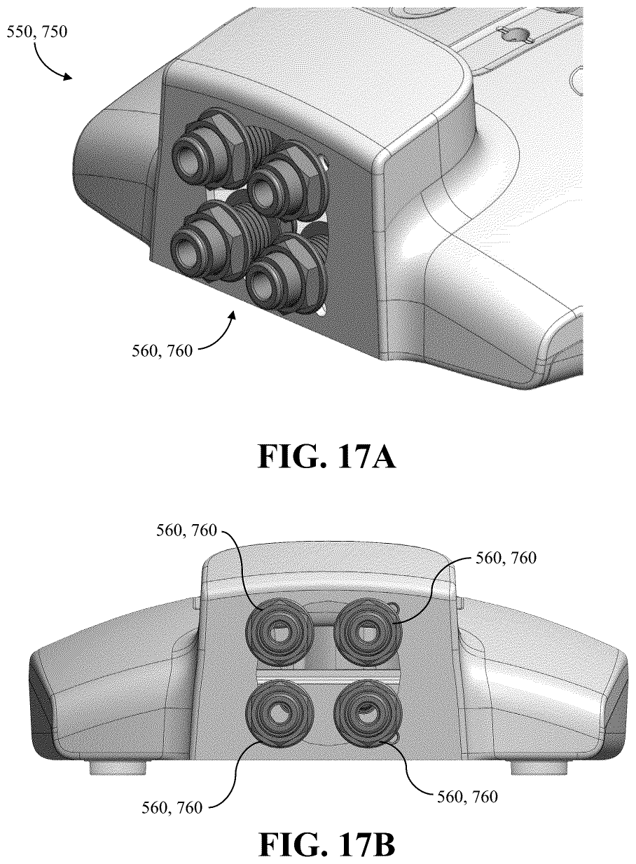

Referring now generally to FIGS. 16A-17B, embodiments of stimulation delivery units described herein, for example units 550, 750, may include a tubing interface 561, 761 defined in connector ridge 564, 764, the tubing interface 561, 761 being configured to receive a tubing coupling 560, 760 attached to tubing. Tubing interface 561, 761 may comprise any suitable connection pattern for receiving tubing coupling 560, 760 (i.e., FIG. 16A illustrates a six-prong tubing interface 561, 761 whereas FIG. 17A illustrates four individual interfaces configured to receive separate tubing couplings 560, 760). Tubing coupling 560, 760 may be couplable to tubing interface 561, 761 by tightening and detachable from interface 561, 761 by untightening.

Referring specifically to FIG. 16C, a cross-section view of stimulation delivery units 550, 750, for example, illustrates the connection between tubing coupling 560, 760 and tubing interface 561, 761 and the interlacing between the plurality of apertures 553a, 753a and the plurality of tubing connectors 553b, 753b within housing 552, 752. The embodiment shown in FIG. 16C also illustrates the position of the plurality of air tubes 562, 726 within housing 552, 752. In operation, air can be directed through tubing coupling 560, 760 and tubing interface 561, 761 and into the plurality of air tubes 562, 762. The plurality of air tubes 562, 762 can then direct air through the plurality of tubing connectors 553b, 753b and out of the plurality of apertures 553a, 753a toward a user's hand, fingers, and/or forearm for stimulation therapy.

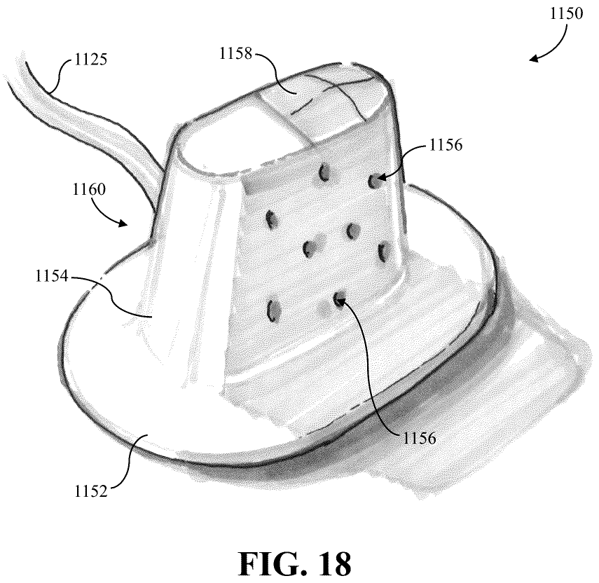

Referring now to FIG. 18, an alternative design for a stimulation delivery unit 1150 according to an embodiment of the present disclosure may include tubing 1125 couplable to a lower housing 1152, an upper housing 1154 couplable to and extending above the lower housing 1152, a plurality of apertures 1156 defined within the upper housing 1154, a pad 1158 couplable to a top surface of the upper housing 1154, and a tubing coupling 1160 for receiving tubing 1125. Stimulation delivery unit 1150 is intended to function similarly to previously described therapy units, specifically directing air through the plurality of apertures 1156 and onto the hand, fingers, and/or forearm of a user. In operation, a user may grasp the upper housing 1154 while placing their thumb or another finger on the pad 1158. The user may rest the remaining portion of their hand, fingers, and/or forearm on the lower housing 1152 for comfort during stimulation therapy. Stimulation delivery unit 1150 may be made from a molded polymer such as polyethylene or polypropylene.

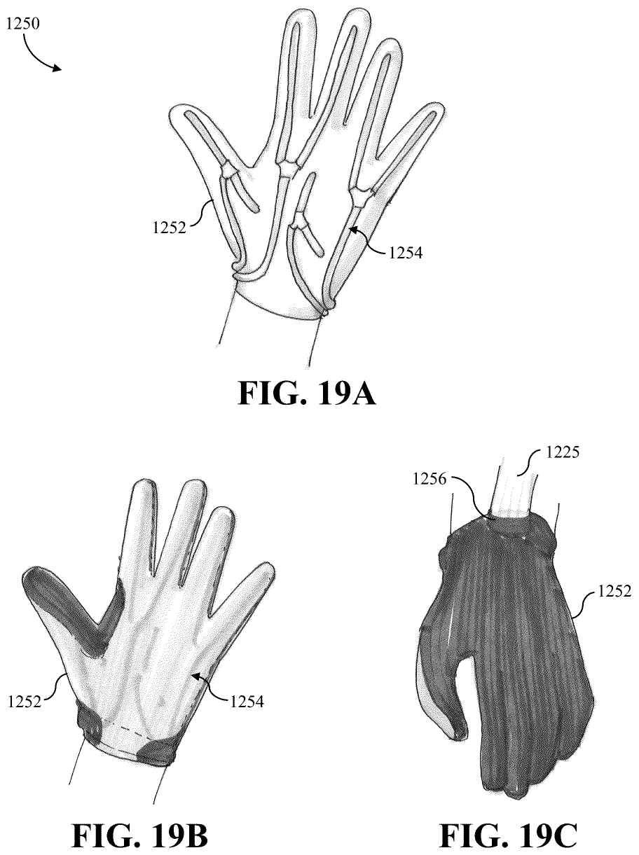

Referring now generally to FIGS. 19A-19C, an alternative design for a stimulation delivery unit 1250 according to an embodiment of the present disclosure may include tubing 1225 couplable to a tubing coupling 1256 connected to a wearable glove 1252. The wearable glove 1252 may include a plurality of air tubes 1254 for transporting air throughout the defined geometry of the wearable glove. Stimulation delivery unit 1250 is intended to function similarly to previously described therapy units, specifically directing air through the plurality of air tubes 1254 and onto the hand and/or fingers of a user. In operation, a user can apply the wearable glove 1252 to either hand and can begin stimulation therapy using the methods described previously.

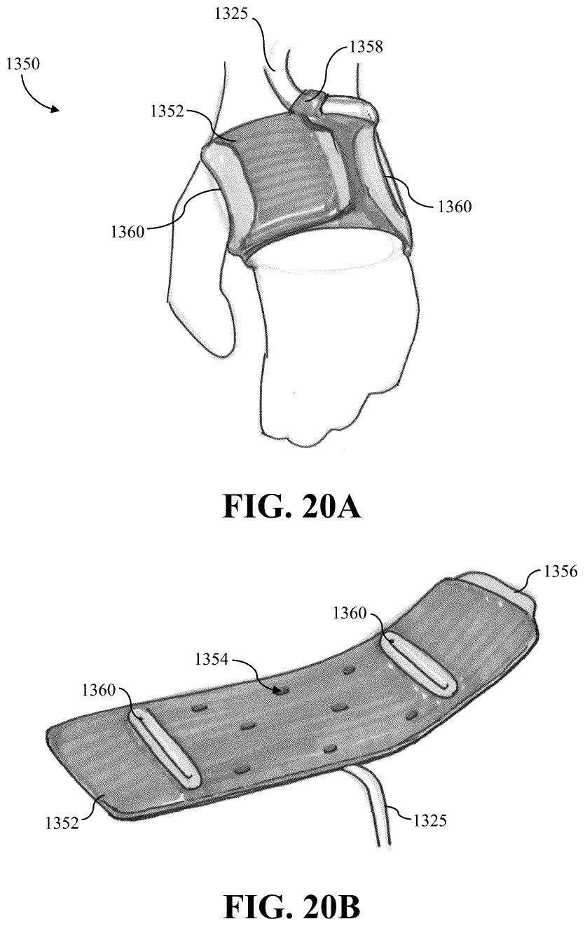

Referring now generally to FIGS. 20A and 20B, an alternative design for a stimulation delivery unit 1350 according to an embodiment of the present disclosure may include tubing 1325 couplable to a tubing coupling 1358 connected to a wrap pad 1352. The wrap pad 1352 may include a plurality of apertures 1354 dispersed on an inner pad side between a pair of defined thumb openings 1360. A first end of the wrap pad 1352 may include a pad coupling mechanism 1356 (e.g., hook and loop attachment, fasteners, adhesives, etc.) configured to connect to a second end to secure the wrap pad 1352 around a user's hand as illustrated in FIG. 20A. Stimulation delivery unit 1350 is intended to function similarly to previously described therapy units, specifically directing air through the plurality of apertures 1354 and onto the hand of a user. In operation, a user can apply the wrap pad 1352 to their hand by placing the little, ring, middle, and index fingers through the opening formed by the first end and second end connection. The thumb may be placed within one of the thumb openings 1360 depending on whether the left hand or the right hand is used (i.e., separate thumb opening 1360 for each hand). Stimulation therapy can be performed using the methods described previously.

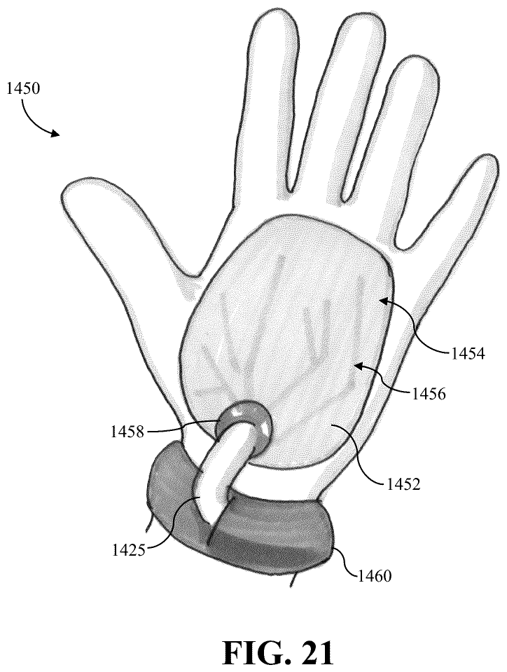

Referring now to FIG. 21, an alternative design for a stimulation delivery unit 1450 according to an embodiment of the present disclosure may include tubing 1425 couplable to a tubing coupling 1458 connected to an outer patch 1452. Outer patch 1452 may include an inner hydrogel 1454 and a plurality of air tubes 1456 disposed within patch 1452. Outer patch 1452, which may be a non-woven material, can be applied to a user's hand as illustrated in FIG. 21 with inner hydrogel 1454 being in direct contact with the user's skin. Stimulation delivery unit 1450 is intended to function similarly to previously described therapy units, specifically directing air through the plurality of air tubes 1456 and onto the hand of a user. Stimulation delivery unit 1450 may further include a tubing wrap pad 1460 for securing a portion of the tubing 1425 to a user's wrist, for example.

Embodiments of peripheral sensory stimulation systems and stimulation delivery units described herein are configured for use at any time convenient for the user. For example, users with insomnia may choose to use the present embodiments before going to bed. Users with anxiety or depression may use the present embodiments in the morning, afternoon, or before going to bed, for example. The disclosed embodiments advantageously decrease symptoms associated with various disorders including anxiety, depression, traumatic brain injury, PTSD, and insomnia, while also increasing user relaxation and satisfaction during treatment.

Various embodiments of systems, devices, and methods have been described herein. These embodiments are given by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill will recognize that the subject matter hereof may comprise fewer features than illustrated in any individual embodiment. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill. Moreover, elements of one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. A peripheral sensory stimulation system, comprising:
a control module;
a power source configured to provide electrical power to the control module; and
a stimulation delivery unit coupled to the control module via tubing, the stimulation delivery unit configured to receive a human appendage to provide stimulation therapy for the treatment of a neurological disorder, wherein the stimulation delivery unit provides stimulation therapy to a user by directing air out of a plurality of apertures and onto the human appendage, the pattern of delivered air being controlled by the control module.

2. The peripheral sensory stimulation system of claim 1, wherein the control module comprises an integrated air compressor configured to deliver compressed air to the stimulation delivery unit.

3. The peripheral sensory stimulation system of claim 1, wherein the human appendage is at least one of a hand, one or more fingers, a forearm, and a foot.

4. The peripheral sensory stimulation system of claim 1, wherein the stimulation delivery unit includes an upper housing separated from a lower housing by a gasket, the upper housing defining the plurality of apertures through which air is provided.

5. The peripheral sensory stimulation system of claim 1, wherein a top surface of the stimulation delivery unit has a convex shape to match the convex shape of a human hand.

6. The peripheral sensory stimulation system of claim 1, wherein the stimulation delivery unit includes a chamber configured to receive the human appendage, the chamber having a floor pad that the human appendage can rest on during stimulation therapy.

7. The peripheral sensory stimulation system of claim 1, wherein the control module is configured to deliver a pattern of pulsed air bursts to the stimulation delivery unit, the control module further configured to control the degree of stimulation via the pattern of pulsed air bursts.

8. The peripheral sensory stimulation system of claim 1, wherein the control module is configured to deliver a pattern of continuous air to the stimulation delivery unit, the control module further configured to control the degree of stimulation via the pattern of continuous air.

9. The peripheral sensory stimulation system of claim 1, wherein the control module is configured to control the temperature of the pattern of delivered air.

10. A peripheral sensory stimulation system, comprising:
a control module;
a power source configured to provide electrical power to the control module;
a first stimulation delivery unit configured to receive a human appendage within a chamber to provide stimulation therapy for the treatment of a neurological disorder, wherein the first stimulation delivery unit provides stimulation therapy to a user by directing air through a plurality of apertures defined in the chamber and toward the human appendage positioned inside the chamber; and
a second stimulation delivery unit configured to receive a human appendage to provide stimulation therapy for the treatment of a neurological disorder, wherein the second stimulation delivery unit provides stimulation therapy to a user by directing air out of a plurality of apertures and onto the human appendage,
wherein the first stimulation delivery unit and the second stimulation delivery unit are configured for interchangeable use with the control module.

11. The peripheral sensory stimulation system of claim 10, wherein the control module comprises an integrated air compressor configured to deliver compressed air to the first stimulation delivery unit and the second stimulation delivery unit.

12. The peripheral sensory stimulation system of claim 10, wherein the human appendage is at least one of a hand, one or more fingers, a forearm, and a foot.

13. The peripheral sensory stimulation system of claim 10, wherein the second stimulation delivery unit includes an upper housing separated from a lower housing by a gasket, the upper housing defining the plurality of apertures through which air is provided.

14. The peripheral sensory stimulation system of claim 10, wherein a top surface of the second stimulation delivery unit has a convex shape.

15. The peripheral sensory stimulation system of claim 10, wherein the control module is configured to deliver a pattern of pulsed air bursts to the first and second stimulation delivery units, the control module further configured to control the degree of stimulation via the pattern of pulsed air bursts.

16. The peripheral sensory stimulation system of claim 10, wherein the control module is configured to deliver a pattern of continuous air to the first and second stimulation delivery units, the control module further configured to control the degree of stimulation via the pattern of continuous air.

17. The peripheral sensory stimulation system of claim 10, wherein the control module is configured to control the temperature of the pattern of delivered air.

18. A stimulation delivery unit, comprising:
a housing configured to receive a human appendage of a user, the housing defining a plurality of apertures in contact with a plurality of air tubes, the plurality of apertures being in contact with at least a portion of the human appendage during operation;
a tunnel structure coupled to the housing, the tunnel structure configured to receive at least a portion of the human appendage; and
a tubing coupling configured to receive tubing connected to an external control module, the tubing coupling further configured to transfer air from the tubing into the housing,
wherein air is directed from the tubing coupling into the plurality of air tubes and out of the plurality of apertures to provide stimulation therapy to the human appendage, thereby treating a neurological disorder of a user through peripheral somatosensory stimulation.

19. The stimulation delivery unit of claim 18, wherein the housing is a hollow chamber that receives the human appendage.

20. The stimulation delivery unit of claim 18, wherein the housing includes:
an upper portion having a convex surface, the plurality of apertures being defined by the upper portion;
a lower portion coupled to the upper portion, the lower portion defining a plurality of channels through which air is transported; and
a gasket positioned between the upper portion and the lower portion, the gasket defining a plurality of apertures corresponding to the plurality of apertures defined by the upper portion.
